(19) United States
(12) Patent Application Publication
(10) Pub. No.: US 2025/0114136 A1
(43) Pub. Date: Apr. 10, 2025

(54) ELECTROSURGICAL DEVICES AND SYSTEMS

(71) Applicant: Relign Corporation, Campbell, CA (US)

(72) Inventors: Aaron Germain, San Jose, CA (US); Kevin Christopher Felt, Morgan Hill, CA (US)

(21) Appl. No.: 18/828,603

(22) Filed: Sep. 9, 2024

Related U.S. Application Data
(60) Provisional application No. 63/588,386, filed on Oct. 6, 2023.

(51) Int. Cl. A61B 18/14; A61B 18/00; A61B 90/00

(52) U.S. Cl. CPC A61B 18/148; A61B 90/36; A61B 2018/00202; A61B 2018/00601; A61B 2018/00696; A61B 2018/00982; A61B 2018/1405; A61B 2090/371

(57) ABSTRACT

A medical device for removing tissue of a patient, optionally comprising: an outer shaft having a lumen, a proximal portion and an distal tip portion with an enlarged dimension relative to a diameter of the proximal portion; and a cutter configured to remove the tissue, wherein the cutter is at least partially received by the distal tip portion and has an enlarged dimension relative to the diameter of the proximal portion of the outer shaft.

### CLAIM OF PRIORITY

This application claims the benefit of U.S. Provisional Patent Application Ser. No. 63/588,386, filed on Oct. 6, 2023, the benefit of priority of which is claimed hereby, and which is incorporated by reference herein in its entirety.

### FIELD OF THE INVENTION

The present invention relates generally to apparatuses for surgical procedures such as a total joint arthroplasty and, more particularly, to apparatuses to surgically treat tissue such as bone and soft tissue.

### BACKGROUND

A variety of surgical apparatuses exist for endoscopic cutting and removal of bone including for subacromial decompression, anterior cruciate ligament reconstruction involving notchplasty, and arthroplasty resection of the acromioclavicular joint. Currently, surgeons use arthroplasty shavers and burrs having rotational cutting surfaces to remove hard and soft tissue in such procedures.

To promote efficiency, endoscopic tool systems including a reusable handpiece and a selection of interchangeable tool probes having different working ends are available. Individual working ends may each have two or more functionalities, such as soft tissue removal and hard tissue resection, fluid removal and imaging, so such tools systems can provide dozens of specific functionalities, providing great flexibility. Typically multiple different tools must be utilized during surgery including different tools to perform coagulation and cutting. This is particularly the case with total joint arthroplasty procedures where multiple tools with different functionality are used.

Arthroplasty procedures on the hip and shoulder require precise tool positioning due to the challenging anatomy of the hip and shoulder joints. For example, in hip arthroscopy, bone and soft tissue resection occurs at the distal tip, at a location which is difficult to access. In shoulder arthroscopy, the resection surfaces are nearly parallel to the device shaft which makes cutting challenging.

### Overview

There is a need for an arthroplasty instrument that can meet the challenging anatomy of the hip and shoulder joints. The present inventor proposes an arthroplasty instrument having a unique distal tip geometry that can accommodate the challenging anatomy of the hip and shoulder joints to allow for more efficient removal of tissue in these joints. In particular, the present inventor proposes an end effector geometry for the distal end of the instrument that includes a ball-shaped cutting tip that improves cutting angle, access to anatomy to be cut, device positioning within the joint, and efficiency during challenging hip and shoulder arthroscopy procedures.

The present inventor has developed improved surgical apparatuses that are instruments with integrated function for soft tissue resection, radiofrequency (RF) ablation, fluid removal, image capture, anatomy illumination and bone-cutting in a single instrument.

The present inventor has also developed a surgical apparatus with further capabilities including an instrument at a distalmost end that can manipulate tissue prior to or after resection. The present inventor further has developed a system wherein a reusable or other handpiece may be removably connected to a replaceable, usually disposable, probe while permitting the various functions discussed above while allowing for vacuum aspiration of fluids including tissue debris through a probe shaft and outwardly through the handpiece without interfering with the electrical and/or mechanical operation of the surgical system to deliver radiofrequency (RF) current to the probe. The present inventor contemplates the surgical apparatuses and systems can reduce costs by eliminating multiple surgical tools, reduce surgical complexity particularly in regards to hip and shoulder procedures and improve surgical efficiency among other benefits. The probes and/or instruments disclosed herein can be configured to be reusable or can be configured to be disposable after the surgery. Thus, the examples provided herein (e.g., reusable v. disposable) are not intended as limiting but are merely provided for exemplary purposes.

### Description of the Background Art

Relevant commonly owned patent publications include: U.S. Pat. Nos. 11,065,023; 11,172,953; US 2018-0303509; US 2019-0008541; US 2019-0059983; US 2019-0134279; US 2019-0021788; US 2018-0317957; US 2019-0008538; US 2019-0083121; US 2018-0263649; US 2017-0290602 and US 2019-0015151, the full disclosures of each of which are incorporated herein by reference.

The following, non-limiting examples, detail certain aspects of the present subject matter to solve the challenges and provide the benefits discussed herein, among others.

Example 1 is a medical device for removing tissue of a patient, optionally comprising: an outer shaft having a lumen, a proximal portion and an distal tip portion with an enlarged dimension relative to a diameter of the proximal portion; and a cutter configured to remove the tissue, wherein the cutter is at least partially received by the distal tip portion and has an enlarged dimension relative to the diameter of the proximal portion of the outer shaft.

In Example 2, the subject matter of Example 1 optionally includes, wherein the cutter is configured to provide the medical device with a positive approach angle to the tissue with an outer surface or one or more edges of the cutter forming an acute inclined angle with an outer surface of the outer shaft.

In Example 3, the subject matter of Examples 1-2 optionally includes, wherein the cutter is rotatable relative to the outer shaft and includes a first side with an electrode having a radius of curvature, wherein the electrode is configured to provide radiofrequency energy to the tissue, and a second side with one or more features for mechanically cutting the tissue.

In Example 4, the subject matter of Example 3 optionally includes, wherein the one or more features include a plurality of resection teeth along a first edge of the second side and a blade having a radius of curvature along a second opposing edge of the second side.

In Example 5, the subject matter of Example 4 optionally includes, wherein the one or more features include a tissue manipulation component at a distalmost end of the cutter.

In Example 6, the subject matter of Examples 1-5 optionally includes, wherein the cutter forms an opening to the lumen, wherein the opening has a maximum dimension that is smaller than a cross-sectional area of the lumen.

In Example 7, the subject matter of Examples 1-6 optionally includes, wherein the outer shaft at the distal tip portion has a hemispherical dish shape.

In Example 8, the subject matter of Examples 1-7 optionally includes, wherein the cutter has a radius of curvature along substantially an entirety of an outer surface thereof.

In Example 9, the subject matter of Examples 1-8 optionally includes, wherein the cutter has a truncated spheroid shape along an outer surface thereof.

In Example 10, the subject matter of Examples 1-9 optionally includes, wherein the cutter is nested in and captured by the distal tip portion such that the cutter is substantially prevented from proximal-distal movement relative to the distal tip portion of the outer shaft.

In Example 11, the subject matter of Examples 1-10 optionally includes, a first visualization assembly coupled to the proximal portion of the outer shaft, wherein the first visualization assembly includes one or more cameras; and a second visualization assembly coupled to the distal tip portion, wherein the second visualization assembly includes one or more cameras.

In Example 12, the subject matter of Example 11 optionally includes, wherein at least one of the first visualization assembly and the second visualization assembly includes a light source.

In Example 13, the subject matter of Examples 11-12 optionally includes, wherein the first visualization assembly has a first field of view that includes at least a portion of the cutter and the second visualization assembly has a second field of view that extends distal of the distal tip portion.

In Example 14, the subject matter of Examples 11-13 optionally includes, wherein the first visualization assembly has a first field of view is offset to a first lateral side of a longitudinal axis of the outer shaft and the second visualization assembly has a second field of view that is offset to a second opposing lateral side of the outer shaft.

In Example 15, the subject matter of Examples 13-14 optionally includes, a display apparatus; and a controller electronically coupled to the display apparatus, the first visualization assembly and the second visualization assembly, wherein the controller is configured control the first visualization assembly and the second visualization assembly and the display apparatus to cause the display apparatus to at least one of: change between the first field of view and the second field of view, provide both the first field of view and the second field of view simultaneously or provide a three-dimensional composite view based upon the first field of view and the second field of view sharing substantially a same focal point.

In Example 16, the subject matter of Example 15 optionally includes, wherein the change between the first field of view and the second field of view is determined by the controller based upon an operation mode of the medical device.

In Example 17, the subject matter of Examples 15-16 optionally includes, wherein the first visualization assembly includes a first one or more light sources and the second visualization assembly includes a second one or more light sources, wherein the controller is configured to control the first one or more light sources and the second one or more lights sources to be one or more of: activated to produce light at a same time; activated in sequence over a period of time; activated to produce light at the same time but with the first one or more light sources having a brightness that differs from a brightness of the second one or more light sources.

Example 18 is a medical device for removing tissue of a patient, optionally comprising: an outer shaft having a lumen, a proximal portion and a distal tip portion with a hemispherical dish shape that has a dimension that is enlarged relative to a diameter of the proximal portion; and a cutter configured to remove the tissue, wherein the cutter is at least partially nested in the distal tip portion and captured by the distal tip portion such that the cutter is substantially prevented from proximal-distal movement relative to the distal tip portion of the outer shaft.

In Example 19, the subject matter of Example 18 optionally includes, wherein the cutter has an enlarged dimension relative to a diameter of the proximal portion of the outer shaft.

In Example 20, the subject matter of Examples 18-19 optionally includes, wherein the cutter is configured to provide the medical device with a positive approach angle to the tissue with an outer surface or one or more edges of the cutter forming an acute inclined angle with an outer surface of the outer shaft.

In Example 21, the subject matter of Examples 18-20 optionally includes, wherein the cutter is rotatable relative to the outer shaft and includes a first side with an electrode having a radius of curvature, wherein the electrode is configured to provide radiofrequency energy to the tissue, and a second side with one or more features for mechanically cutting the tissue.

In Example 22, the subject matter of Example 21 optionally includes, wherein the one or more features include a plurality of resection teeth along a first edge of the second side and a blade having a radius of curvature along a second opposing edge of the second side.

In Example 23, the subject matter of Example 22 optionally includes, wherein the one or more features include a tissue manipulation component at a distalmost end of the cutter.

In Example 24, the subject matter of Examples 18-23 optionally includes, wherein the cutter forms an opening to the lumen, wherein the opening has a maximum dimension that is smaller than a cross-sectional area of the lumen.

In Example 25, the subject matter of Examples 18-24 optionally includes, wherein the cutter has a radius of curvature along substantially an entirety of an outer surface thereof.

In Example 26, the subject matter of Examples 18-25 optionally includes, wherein the cutter has a truncated spheroid shape along an outer surface thereof.

In Example 27, the subject matter of Examples 18-26 optionally includes, a first visualization assembly coupled to the proximal portion of the outer shaft, wherein the first visualization assembly includes one or more cameras; and a second visualization assembly coupled to the distal tip portion, wherein the second visualization assembly includes one or more cameras.

In Example 28, the subject matter of Example 27 optionally includes, wherein at least one of the first visualization assembly and the second visualization assembly includes a light source.

In Example 29, the subject matter of Examples 27-28 optionally includes, wherein the first visualization assembly has a first field of view that includes at least a portion of the cutter and the second visualization assembly has a second field of view that extends distal of the distal tip portion.

In Example 30, the subject matter of Examples 27-29 optionally includes, wherein the first visualization assembly has a first field of view is offset to a first lateral side of a longitudinal axis of the outer shaft and the second visualization assembly has a second field of view that is offset to a second opposing lateral side of the outer shaft.

In Example 31, the subject matter of Examples 29-30 optionally includes, a display apparatus; and a controller electronically coupled to the display apparatus, the first visualization assembly and the second visualization assembly, wherein the controller is configured control the first visualization assembly and the second visualization assembly and the display apparatus to cause the display apparatus to at least one of: change between the first field of view and the second field of view, provide both the first field of view and the second field of view simultaneously or provide a three-dimensional composite view based upon the first field of view and the second field of view sharing a same focal point.

In Example 32, the subject matter of Example 31 optionally includes, wherein the change between the first field of view and the second field of view is determined by the controller based upon an operation mode of the medical device.

In Example 33, the subject matter of Examples 31-32 optionally includes, wherein the first visualization assembly includes a first one or more light sources and the second visualization assembly includes a second one or more light sources, wherein the controller is configured to control the first one or more light sources and the second one or more light sources to be one or more of: activated to produce light at a same time; activated in sequence over a period of time; activated to produce light at the same time but with the first one or more light sources having a brightness that differs from a brightness of the second one or more light sources.

Example 34 is a medical device for removing tissue of a patient, optionally comprising: an outer shaft having a lumen, a proximal portion and a distal tip portion with an enlarged dimension relative to a diameter of the proximal portion; and a cutter configured to remove the tissue, wherein the cutter is at least partially received by the distal tip portion and is configured to provide the medical device with a positive approach angle to the tissue with an outer surface or one or more edges of the cutter forming an acute inclined angle with an outer surface of the outer shaft.

In Example 35, the subject matter of Example 34 optionally includes, wherein the cutter has an enlarged dimension relative to a diameter of the proximal portion of the outer shaft.

In Example 36, the subject matter of Examples 34-35 optionally includes, wherein the cutter is rotatable relative to the outer shaft and includes a first side with an electrode having a radius of curvature, wherein the electrode is configured to provide radiofrequency energy to the tissue, and a second side with one or more features for mechanically cutting the tissue.

In Example 37, the subject matter of Example 36 optionally includes, wherein the one or more features include a plurality of resection teeth along a first edge of the second side and a blade having a radius of curvature along a second opposing edge of the second side.

In Example 38, the subject matter of Example 37 optionally includes, wherein the one or more features include a tissue manipulation component at a distalmost end of the cutter.

In Example 39, the subject matter of Examples 34-38 optionally includes, wherein the cutter forms an opening to the lumen, wherein the opening has a maximum dimension that is smaller than a cross-sectional area of the lumen.

In Example 40, the subject matter of Examples 34-39 optionally includes, wherein the cutter has a radius of curvature along substantially an entirety of an outer surface thereof.

In Example 41, the subject matter of Examples 34-40 optionally includes, wherein the cutter has a truncated spheroid shape along the outer surface thereof.

In Example 42, the subject matter of Examples 34-41 optionally includes, a first visualization assembly coupled to the proximal portion of the outer shaft, wherein the first visualization assembly includes one or more cameras; and a second visualization assembly coupled to the distal tip portion, wherein the second visualization assembly includes one or more cameras.

In Example 43, the subject matter of Example 42 optionally includes, wherein at least one of the first visualization assembly and the second visualization assembly includes a light source.

In Example 44, the subject matter of Examples 42-43 optionally includes, wherein the first visualization assembly has a first field of view that includes at least a portion of the cutter and the second visualization assembly has a second field of view that extends distal of the distal tip portion.

In Example 45, the subject matter of Examples 42-44 optionally includes, wherein the first visualization assembly has a first field of view is offset to a first lateral side of a longitudinal axis of the outer shaft and the second visualization assembly has a second field of view that is offset to a second opposing lateral side of the outer shaft.

In Example 46, the subject matter of Examples 44-45 optionally includes, a display apparatus; and a controller electronically coupled to the display apparatus, the first visualization assembly and the second visualization assembly, wherein the controller is configured control the first visualization assembly and the second visualization assembly and the display apparatus to cause the display apparatus to at least one of: change between the first field of view and the second field of view, provide both the first field of view and the second field of view simultaneously or provide a three-dimensional composite view based upon the first field of view and the second field of view sharing a same focal point.

In Example 47, the subject matter of Example 46 optionally includes, wherein the change between the first field of view and the second field of view is determined by the controller based upon an operation mode of the medical device.

In Example 48, the subject matter of Examples 46-47 optionally includes, wherein the first visualization assembly includes a first one or more light sources and the second visualization assembly includes a second one or more light sources, wherein the controller is configured to control the first one or more light sources and the second one or more light sources to be one or more of: activated to produce light at a same time; activated in sequence over a period of time; activated to produce light at the same time but with the first one or more light sources having a brightness that differs from a brightness of the second one or more light sources.

Example 49 is a surgical tissue shaving device optionally comprising: an outer shaft; an inner tube rotatably disposed within the outer shaft; and a ball-shaped tip at a distal end of the inner tube, the ball-shaped tip having a larger outer diameter than the outer shaft; and a plurality of cutting edges on the ball-shaped tip configured to resect bone.

In Example 50, the subject matter of Example 49 optionally includes, wherein the ball-shaped tip comprises an electrode and a ceramic insulator configured to provide ablation.

In Example 51, the subject matter of Examples 49-50 optionally includes, wherein the ball-shaped tip comprises a non-cutting manipulation tooth configured to manipulate soft tissue.

In Example 52, the subject matter of Examples 49-51 optionally includes, wherein the ball-shaped tip comprises an inner diameter smaller than subsequent inner diameters along the inner tube to restrict tissue entry.

In Example 53, the subject matter of Examples 49-52 optionally includes, wherein the ball-shaped tip comprises a recess on a proximal end configured to mate with a distal end of the outer shaft to prevent axial movement of the ball-shaped tip.

Example 54 is at least one machine-readable medium including instructions that, when executed by processing circuitry, cause the processing circuitry to perform operations to implement of any of Examples 1-53.

Example 55 is an apparatus comprising means to implement of any of Examples 1-53.

Example 56 is a system to implement of any of Examples 1-53.

Example 57 is a method to implement of any of Examples 1-53.

In Example 58, the devices of any one or any combination of Examples 1-57 can optionally be configured such that all elements or options recited are available to use or select from.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1 is an exploded view of an arthroplasty cutting system that includes an electrosurgical device having reusable handpiece with a detachable single-use probe according to an example of the present disclosure.

FIG. 2 is side view of the probe of FIG. 1 according to an example of the present disclosure.

FIG. 2A is a side view of another example of a probe having and angulated distal (working) end according to an example of the present disclosure.

FIG. 3 is an enlarged perspective view of a distal (working) end of the probe of FIG. 2 with a cutter in a first position according to an example of the present disclosure.

FIG. 4 is an enlarged perspective view of the distal (working) end of the probe of FIG. 2 with the cutter in a second position.

FIG. 5 is a first cross-sectional view of the distal (working) end of the probe of FIG. 2.

FIG. 6 is a second cross-sectional view of the distal (working) end of the probe of FIG. 2.

FIG. 7A is a side plan view of a distal (working) end of a probe according to another example of the present disclosure.

FIG. 7B is a perspective view of the distal (working) end of the probe of FIG. 7A.

FIGS. 8A and 8B are different side plan views of the distal (working) end of the probe of FIG. 2 showing the cutter having a positive approach angle relative to a shaft according to an example of the present disclosure.

FIGS. 9A-9C show example distal end cutters that have a negative approach angle relative to a shaft according to examples of the present disclosure.

FIG. 10 shows a surgical device with a distal end cutter with a negative approach angle similar to those of the cutters of examples of FIGS. 9A-9C in a hip joint according to an example of the present disclosure.

FIG. 11 shows a surgical device with a distal end cutter with a negative approach angle similar to those of the cutters of examples of FIGS. 9A-9C in a hip joint according to an example of the present disclosure.

FIG. 12 is side view of a prove having a first visualization assembly and a second visualization assembly according to an example of the present disclosure.

FIG. 13 is an enlarged perspective view of a distal (working) end of the probe of FIG. 12 with the first visualization assembly coupled to an outer shaft and the second visualization assembly coupled to a distal tip portion.

FIG. 14 is a view from a distal perspective of distal (working) end of the probe of FIG. 12 along with a schematic representation of a controller; wherein the controller is controlling various functions of the first visualization assembly and the second visualization assembly according to examples of the present disclosure.

FIGS. 15-17 schematically illustrate the controller controlling various display functions on a display using imaging from one or both of first visualization assembly and the second visualization assembly.

FIG. 18 illustrates a block diagram of an example machine upon which any one or more of the techniques, apparatuses, systems or methods discussed herein may perform in accordance with at least one example of this disclosure.

### DETAILED DESCRIPTION OF THE INVENTION

The present invention relates to electrosurgical devices that have various functions including soft tissue resection, RF ablation, image capture, anatomy illumination, fluid removal and bone-cutting in a single instrument. Additionally, the electrosurgical devices include an improved cutter as further discussed herein. Several embodiments of the devices will now be described to provide an overall understanding of the principles of the form, function and methods of use. In general, the present disclosure provides for electrosurgical devices that can be used as arthroplasty tools including for total joint arthroplasty such as for the hip or shoulder joint. The arthroplasty tools are typically disposable and are configured for detachable coupling to a non-disposable handpiece. This description of the general principles of this invention is not meant to limit the inventive concepts in the appended claims.

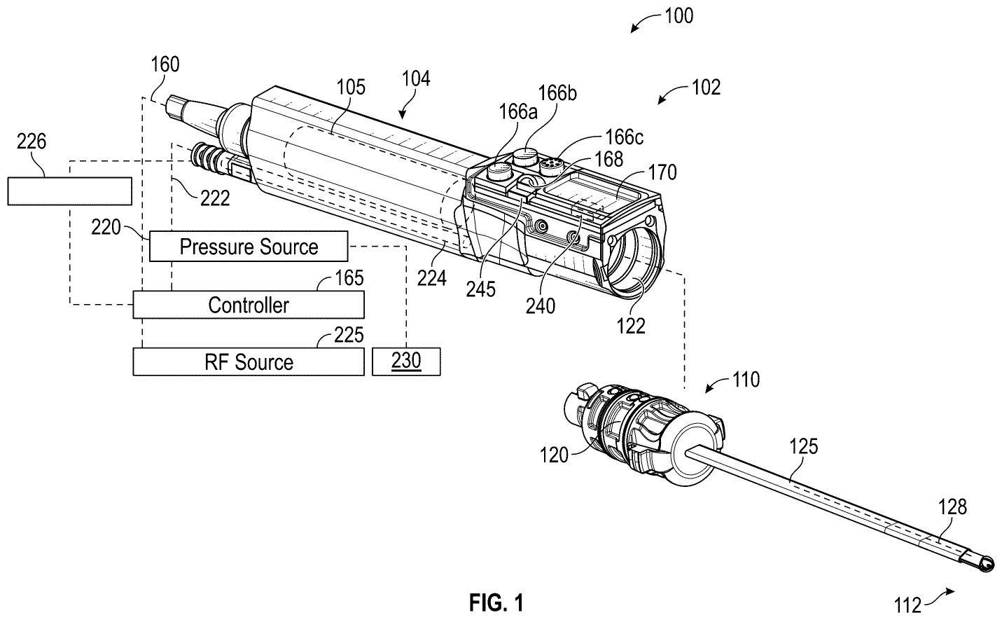

In one example shown in FIG. 1, an arthroplasty system 100 of the present invention provides an electrosurgical apparatus 102 having a handpiece 104 with motor drive 105 and a probe 110 with a proximal hub 120 that can be received by receiving passageway 122 in the handpiece 104. In one aspect, the probe 110 has a distal (working) end 112 that carries soft tissue cutting mechanism(s), bone cutting mechanism(s) and RF electrode configured for use in many arthroplasty surgical applications, including but not limited to treating bone in shoulders, knees, hips, wrists, ankles and the spine.

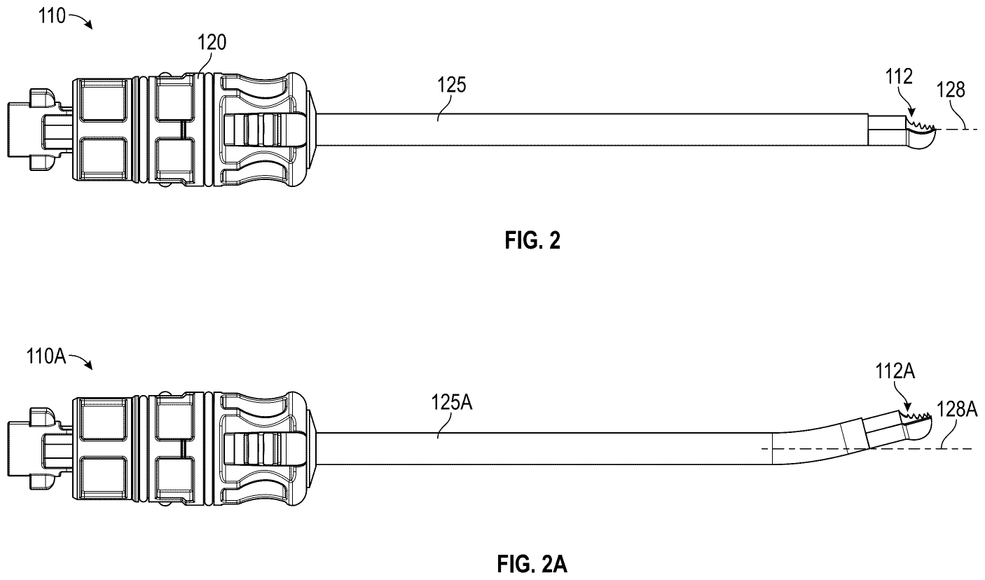

As can be seen in FIGS. 1 and 2, the probe 110 is attachable to and detachable from the handpiece 104. In FIGS. 1 and 2, the probe 110 has a shaft 125 extending along longitudinal axis 128. A distal portion of the shaft 125 including the distal end 112. The shaft 125 can be somewhat flexible or rigid as desired and can house various components that can extend from the hub 120 to the distal end 112 as further discussed. Thus, the shaft 125 can comprise tube or outer sleeve with components such as wires, flow channels, additional shafts, and the like passing therethrough. The shaft 125 extends from the hub 120 (located at a proximal end of the shaft 125) to the distal end 112. The shaft 125 can be coupled in a fixed manner to the hub 120 which can be an injection molded plastic, for example, with the shaft 125 insert molded therein. One or more components can pass through the shaft 125 including to provide RF energy to the electrode(s), provide for image capture, illumination, fluid removal, provide for mechanical cutting or the like. The motor drive 105 (FIG. 1) can be used to rotate a cutter at the distal end 112 as further described to perform mechanical cutting of tissue or switch to application of RF energy with the probe 110.

In FIG. 1, it can be seen that the handpiece 104 is operatively coupled by electrical cable 160 to a controller 165 which can control the motor drive 105, communication with a pressure source 220, and communication with the RF source 225. Actuator buttons 166a, 166b, 166c, etc. on the handpiece 104 (sometimes called a handle herein) can be used to select operating modes (e.g., actuate the motor to switch from RF application to mechanical cutting), select current strength for RF, flow control, or the like. In one variation, a joystick 168 can be moved forward and backward to adjust the rotational speed of motor or other function such as to extend or retract an electrode (discussed subsequently). An LCD screen 170 can provided in the handpiece 104 for displaying operating parameters, such as mode of operation, etc.

It can be understood from FIG. 1 that the system 100 and handpiece 104 can be configured for use with various disposable probes which can be designed for various different functions and procedures. Some of the probes can utilize the motor drive 105, for example, and some may not. These probes are various described in the various applications incorporated by reference with the U.S. Application Publications noted above.

FIG. 1 further shows that the system 100 also includes a pressure source 220 such as a negative pressure source coupled to aspiration tubing 222 which communicates with a flow channel 224 in handpiece 104 and can cooperate with one or more tubes of the probe 110. The system 100 includes the RF source 225 which can be connected to an electrode arrangement of the probe 110. The system 100 can include flow inducing device 226 such as a pump, positive pressure source or the like that passes in fluid communication to the handpiece 104 and to the distal end 112. The flow inducing device 226 (optionally controlled by the controller 165) can allow for flow to the distal end 112 of a fluid such as for application of an irrigating fluid (e.g., saline) utilized during operation of the electrosurgical apparatus 102. The controller 165 and microprocessor therein together with control algorithms are provided to operate and control all functionality, which includes controlling the motor drive 105, the RF source 225, the flow inducing device 226, illuminating device, and the negative pressure source 220 which can aspirate fluid including tissue debris to collection reservoir 230.

As can be understood from the above description of the system 100, the electrosurgical device 102 and handpiece 104, the controller 165 and controller algorithms can be configured to perform and automate many tasks to provide for system functionality. In a first aspect, controller algorithms are needed for device identification so that when any of the different probes types are coupled to handpiece 104, the controller 165 will recognize the probe type and then select algorithms for operating the motor drive 105, RF source 225, flow inducing device 226, negative pressure source 220, etc. as is needed for the particular probe. In a second aspect, the controller can be configured with algorithms that identify whether the probe is coupled to the handpiece 104 in a particular orientation relative to the handpiece, wherein each orientation requires a different subset of the operating algorithms.

Referring to FIG. 1, the handpiece 104 can carry a first Hall effect sensor 240 in a distal region of the handpiece 104 adjacent the receiving passageway 122 that receives the hub 120 of probe 110. The handpiece 104 can carry a second Hall effect sensor 245 adjacent the rotatable drive coupling 150 of the probe 110. The probe 110 can carry a plurality of magnets that interact with the Hall effect sensors 240, 245 to provide multiple control functions in cooperation with controller algorithms, including (i) identification of the type of probe coupled to the handpiece, and (ii) the orientation of the hub 120 relative to the handpiece 104.

The Hall sensor 240 and controller algorithms can be adapted to read the magnetic field strength of the particular magnet(s) in the probe which can be compared to a library of field strengths that correspond to particular probe types. Then, a Hall identification signal can be generated or otherwise provided to the controller 165 to select the controller algorithms for operating the identified probe, which can include parameters for operating the motor drive 105, negative pressure source 220, the flow inducing device 226, power source (e.g., for illumination and other function) and/or RF source 225 as may be required for the probe type. The Hall sensor 240 and associated algorithms look for magnetic field strength regardless of polarity to identify the probe type.

The electrosurgical device 102 can be operated in different modes including mechanical soft tissue resection mode, mechanical bone resection mode and an RF mode. These modes and features facilitating them will be discussed in further detail subsequently. The electrosurgical device 102 can be operated in different RF modes. One mode can deliver RF current in a cutting waveform to thereby create a plasma that ablates tissue. In another RF mode, the controller 165 can include an algorithm that controls RF current in a coagulation waveform.

The controller 165 can include, for example, software, hardware, and combinations of hardware and software configured to execute several functions related to, among others, operation of the system 100 (FIG. 1). The controller 130 can be an analog, digital, or combination analog and digital controller including a number of components. As examples, the controller 165 can include integrated circuit boards or ICB(s), printed circuit boards PCB(s), processor(s), data storage devices, switches, relays, or any other components. Examples of processors can include any one or more of a microprocessor, a controller, a digital signal processor (DSP), an application specific integrated circuit (ASIC), a field-programmable gate array (FPGA), or equivalent discrete or integrated logic circuitry. Commercially available microprocessors can be configured to perform the functions of the controller 165. Various known circuits may be associated with controller 165, including power supply circuitry, signal-conditioning circuitry, actuator driver circuitry (i.e., circuitry powering solenoids, motors, or piezo actuators), and communication circuitry. In some examples, the controller 165 may be positioned on the handpiece 104, while in other examples the controller 165 may be positioned at an off-board location (remote location) relative to the handpiece 104 such as in a control unit.

The controller 165 can include a memory such as memory circuitry. The memory may include storage media to store and/or retrieve data or other information such as, operational algorithms. Storage devices, in some examples can be a computer-readable storage medium. The data storage devices can be used to store program instructions for execution by processor(s) of the controller 165, for example. The storage devices, for example, are used by software, applications, algorithms, as examples, running on and/or executed by the controller 165. The storage devices can include short-term and/or long-term memory and can be volatile and/or non-volatile. Examples of non-volatile storage elements include magnetic hard discs, optical discs, floppy discs, flash memories, or forms of electrically programmable memories (EPROM) or electrically erasable and programmable (EEPROM) memories. Examples of volatile memories include random access memories (RAM), dynamic random-access memories (DRAM), static random-access memories (SRAM), and other forms of volatile memories known in the art.

FIG. 2A shows an example of a distal end 112A of a shaft 125A of a probe 110A where the distal end 112A and distal portion of the shaft 125A is angulated relative to a longitudinal axis 128A of the shaft 125A. The angulation at the distal end of the shaft 125A can improve access to tissue within a joint. The angulated shaft 125A can use a flexible drive shaft for actuation of the mechanical and RF cutting modes discussed further subsequently.

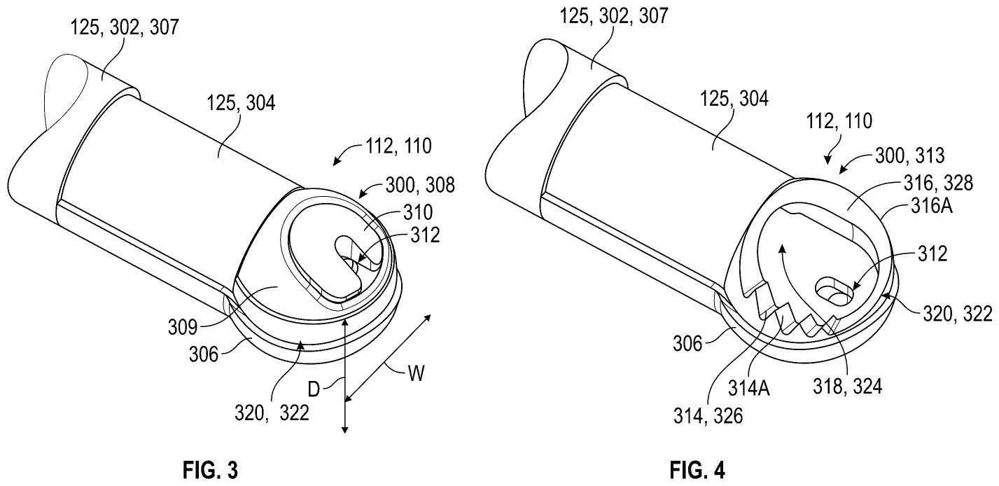

FIG. 3 shows an example of the distal end 112 of the shaft 125 of the probe 110 with a cutter 300 in a first position for operation in the RF mode such as to ablate or coagulate tissue. FIG. 4 shows the distal end 112 of the shaft 125 of the probe 110 with the cutter 300 in a second position for operation in a tissue cutting mode such as to resect soft tissue and/or to resect bone. As shown in FIGS. 3 and 4, the shaft 125 includes an outer shaft 302 and an inner shaft 304. A distal tip portion 306 can be part of the outer shaft 302 or can be a separate component therefrom that is coupled to the outer shaft 302. The distal tip portion 306 and the outer shaft 302 are stationary while the inner shaft 304 can rotate and/or otherwise oscillate as driven by the motor drive 105 (FIG. 1), for example. For simplicity, the distal tip portion 306 may be discussed synonymously herein with the outer shaft 302 and the distal tip portion 306 can simply be referred to as an outer shaft, an outer tip, a tip component, a distal tip, or a capture component herein. The outer shaft 302 can include a proximal portion 307 that can be adjacent to and connected with the distal tip portion 306. The proximal portion 307 can at a distal part of the outer shaft 302 proximal of the distal tip portion 306 and the distal end 112.

Referring first to FIG. 3, the cutter 300 can include a first side 308 with an insulator 309, an electrode 310 and a first port 312. Referring now to FIG. 4, the cutter 300 has been rotated substantially 180 degrees from the first position of FIG. 3 to the second position. The cutter 300 as shown in FIG. 4 includes a second side 313 with a first cutting feature 314 (e.g., teeth 314A), a second cutting feature 316 (e.g., a blade or cutting edge 316A) and a second port 318. A portion of the first port 312 is also shown in FIG. 4.

The inner shaft 304 can be received within the outer shaft 302 and can be movable (e.g., rotatable) relative thereto. The inner shaft 304 can be a cannulated so as to be a tube to provide for inflow and/or outflow of fluid as further discussed herein. The inner shaft 304 can be coupled to the cutter 300 such that the cutter 300 is movable with the inner shaft 304. The distal tip portion 306 can have a generally dish, bowl, hemispherical dish or hemispherical shape having an opening 320 on a first side but being enclosed on a second side. The cutter 300 can include a main body portion 322 configured to at least partially reside in the distal tip portion 306. The main body portion 322 can have a partial dish, bowl, hemispherical dish or truncated spherical shape so as to be moveably received in the distal tip portion 306. Save for the first port 312 and the second port 318, the cutter 300 can have a radius of curvature along substantially an entirety of an outer surface thereof. The cutter 300 can have a truncated spheroid shape along an outer surface thereof due to its configuration. As will be further discussed herein, the cutter 300 can be nested in and captured by the distal tip portion 306 such that the cutter 300 is substantially prevented from proximal-distal movement relative to the distal tip portion 306.

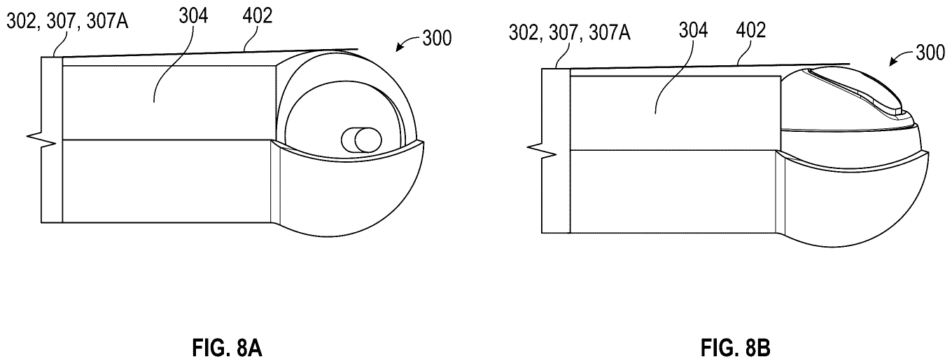

As shown in FIG. 3, the distal tip portion 306 can have an enlarged dimension(s) such as a width W and/or depth D relative to a dimension such as a diameter of the proximal portion 307 of the outer shaft 302. Similarly, the cutter 300 can have an enlarged dimension(s) such as the width W and/or depth D relative to a dimension such as a diameter of the inner shaft 304. In some examples, the cutter 300 can have the enlarged dimension(s) such as the width W and/or depth D relative to a dimension such as the diameter of the proximal portion 307 of the outer shaft 302. The configuration of the cutter 300 and/or the distal tip portion 306 relative to the outer shaft 302 and/or the inner shaft 304 can allow the probe 110 to have a positive approach angle to tissue, which has benefits as further discussed in detail in regards to FIGS. 8A-11.

As shown in FIG. 3, the insulator 309 surrounds a portion of the electrode 310. The insulator 309 has a radius of curvature and can have a dome, hemispherical or truncated hemispherical shape with an opening/passage that receives the electrode 310. The insulator 309 can be coupled to the main body portion 322 of the cutter 300. The insulator 309 can be constructed of or coated by an electrically insulating material such as ceramic, while the main body portion 322 can be constructed of another material such as metal or metal alloy that is electrically conducting. The electrode 310 can be constructed of conductive metal or metal alloy such as tungsten, alloys including tungsten, or the like. The electrode 310 can be configured for bipolar operation alternating between active and return or monopolar operation. A second electrode (not shown) may be utilized at or proximal of the distal end 112. A level of RF energy to the electrode 310 can be controlled as desired for use in coagulation or RF ablation.

The electrode 310 can have a radius of curvature. Such radius of curvature and other dimension(s) can allow the electrode 310 to seat within the insulator 309. Together the insulator 309 and the electrode 310 can form the outer surface of the cutter 300 with the outer surface having substantially a same radius of curvature along the first side 308. The electrode 310 can form at least a portion of the first port 312. The first port 312 can additionally be formed by the insulator 309. The main body portion 322 can have a dome, hemispherical or truncated hemispherical, or truncated spheroid shape and can be received within the generally dish, bowl or hemispherical shape of the distal tip portion 306. The first side 308 can be enclosed save for the first port 312. A major dimension of the electrode 310 can be between. .05 mm and 3.5 mm, for example. The electrode 310, and indeed the insulator 309 and other portions of the cutter 300, can have another shape such as being flat, concave, convex, etc. according to further examples.

Referring now to FIG. 4, in the second position the second side 313 of the cutter 300 is exposed. The second side 313 has an open or scoop type configuration with the second port 318 being a recess surrounded by the first cutting features 314 and the second cutting features 316. The second port 318 can be directly exposed to tissue and can be in communication with the distalmost end of the cutter 300. The second port 318 can be an opening to a suction lumen 324, for example. The first port 312 can communicate with the second side 313 and with the second port 318. The first port 312 can be adjacent and distal of the inner shaft 304. The second side 313 includes the first cutting feature 314 (e.g., the teeth 314A) and the second cutting feature 316 (e.g., blade or cutting edge 316A). The first cutting feature 314 and the second cutting feature 316 can be formed by the main body portion 322 at the second side 313. The first cutting features 314 can be on a first medial-lateral side 326 spaced across the width of the cutter 300 from a second medial-lateral side 328 with the second cutting features 316. This arrangement has the first cutting features 314 on an opposing medial-lateral side of the cutter 300 from the second cutting features 316. However, the second cutting features 316 and/or the first cutting features 314 can extend around proximally, distally, medially and/or laterally to the other of the first cutting features 314 and/or the second cutting features 316 such that the cutter 300 can have substantially continuous cutting features in three dimensions.

The second port 318 and the suction lumen 324 can extend along the shaft 125 and can be in fluid communication with the flow channel 224 (FIG. 1) in handpiece 104 (FIG. 1) and further in communication with the aspiration tubing 222 which communicates with the negative pressure source 220 (FIG. 1). Via the first port 312 and the second port 318, the fluid (e.g., irrigating fluid, blood, tissue debris, smoke, etc.) can be aspirated and pass away from the surgical site. The location of the first and second ports 312 and 318 in FIGS. 3 and 4 is purely exemplary and can be in other locations such as at the distal tip portion 306, etc. The use of additional ports are also contemplated. Although not shown in FIGS. 3 and 4, the device can include one or more ports for outlet of irrigating fluid (e.g., saline) to the surgical area (also called the surgical site herein) allowing the irrigating fluid to pass from the electrosurgical device to the surgical area.

The first cutting features 314 can include the teeth 314A configured for soft tissue resection. The teeth 314A can have differing depths and/or sizes in the depth dimension and other dimension(s) due to a radius of curvature along the edge. The second cutting features 316 (e.g., the blade or cutting edge 316A) can be configured for bone resection. The blade or cutting edge 316A can have a radius of curvature along the second medial-lateral side 328 of the cutter 300. Indeed, the blade or cutting edge 316A can smoothly transition and curve in several dimensions including medial-lateral, depth, and proximal-distal directions as shown in FIG. 4. The blade or cutting edge 316A can be formed by a combination of a chamfered surface with one or more sharp edges, for example.

The present application contemplates the cutter 300 can be rotated by the motor drive or other drive mechanism. The cutter 300 can be rotated (typically at least 3,000 rpm), and the controller (FIG. 1) operatively connects the suction lumen 324 to the negative pressure source and the RF source to the electrode 310. Tissue debris is aspirated through the suction lumen 324 and the cutter 300 can be manipulated within the joint or other anatomical area to remove a surface portion of the targeted tissue and/or to undercut the targeted tissue to thereby remove chips of tissue. The cutter 300 can operate as a high-speed rotating cutter or burr that is operable at rotational speeds ranging from 3,000 rpm to 20,000 rpm.

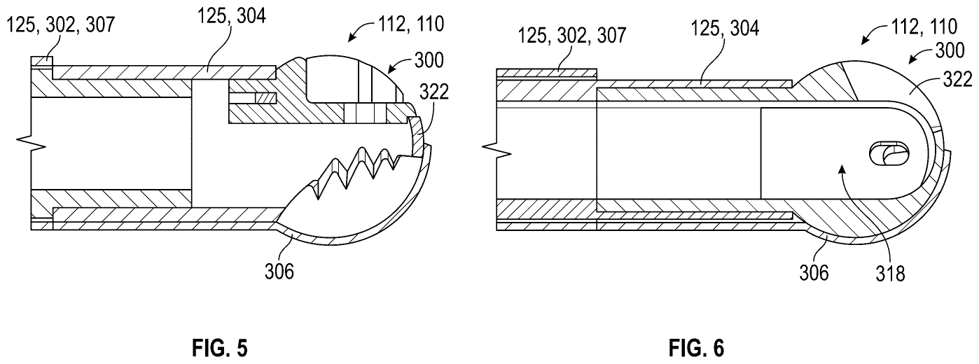

FIGS. 5 and 6 are cross-sectional views of the distal end 112 of the shaft 125 of the probe 110 with the cutter 300, the outer shaft 302, the inner shaft 304, the distal tip portion 306 and the proximal portion 307. FIG. 6 shows the cutter 300 in the process of being rotated from the first position of FIG. 3 to the second position of FIG. 4. FIGS. 5 and 6 show the cutter 300 has a substantially ball or spherical shape with an enlarged dimension(s) relative to the proximal portion 307 of the outer shaft 302 and the inner shaft 304 as previously discussed. As shown in FIG. 6, the main body portion 322 can be thickened in the regions of the first cutting features 314 (FIG. 4) and the second cutting features 316 (FIG. 4) to provide the second port 318 (FIG. 6) with an appropriate dimension(s) such that cut material and other fluid does not clog the suction lumen 324 (FIG. 4). FIGS. 5 and 6 additionally show how the cutter 300 is nested in and captured by the distal tip portion 306 (e.g., rests within and is captured by the hemispherical dish shape) such that the cutter 300 can be substantially prevented from proximal-distal movement relative to the distal tip portion 306. One design difficulty with typical bone cutters is controlling the axial position of the cutting tip when spinning at high RPMs required for bone cutting. Traditional typical devices used soft springs to keep the device pushed forward but allow the cutting tip to float backwards under load. With the present design, because the cutting tip is enlarged relative to the shaft(s) diameter, if the cutting tip were to move backwards it could come into contact with the stationary shaft causing the cutting tip to lock up, shear or another undesired outcome. Thus, the present design captures the cutter 300 so that proximal-distal movement is substantially prevented.

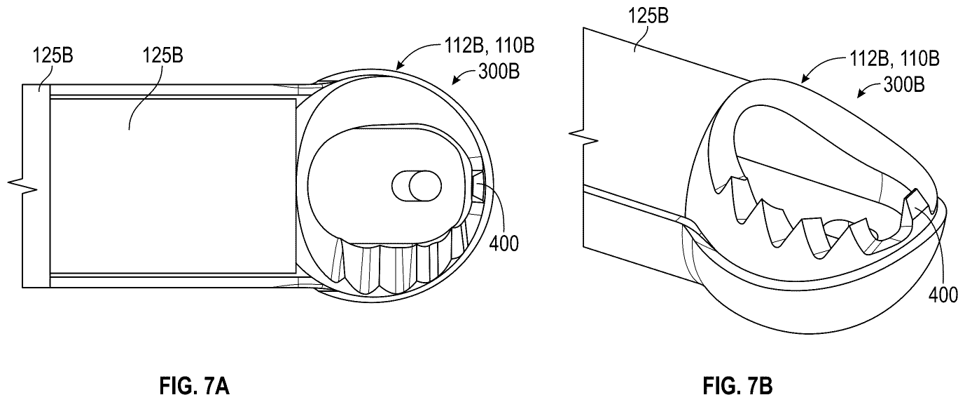

FIGS. 7A and 7B show views of a distal end 112B of a shaft 125B of a probe 110B with a cutter 300B according to another example. The distal end 112B, cutter 300B and the probe 110B have a construct similar to that of those of FIGS. 1-6 discussed previously. The cutter 300B additionally includes a tissue manipulation component 400 (e.g., a tooth) at a distalmost end of the cutter 300B. This tissue manipulation component 400 can be a non-cutting or cutting tooth at the center distalmost tip. When in the cutter 300B is in the second position as shown in FIGS. 7A and 7B, the tissue manipulation component 400 can provide an enlarged edge on which to use the device as a probe. This can be useful in a hip procedure as the labrum needs to be lifted such that a surgeon can bone burr beneath it to prepare the site for anchors. Because the tissue manipulation component 400 can be substantially at the centerline (at the distalmost tip) tissue manipulation component 400 may not needed in resection, and therefore, does not need to be a sharp cutting feature.

FIGS. 8A and 8B illustrate the positive approach angle 402 (indicated by a line) to tissue of different portions of the cutter 300 as regards the proximal portion 307 of the outer shaft 302 and the inner shaft 304. As shown, the positive approach angle to the tissue as measured between an outer surface and/or one or more edges of the cutter 300 can form an acute inclined angle with an outer surface 307A of the outer shaft 302 as shown. The outer surface and/or one or more edges of the cutter 300 can be disposed radially outward of the outer surface 307A of the outer shaft 302. This arrangement can provide for improved access, manipulation and cutting using the cutter 300 within the joint. Specifically, because the outer diameter of the cutter 300 is larger than the outer diameter of the outer shaft 302, the configuration allows the bone cutting features of the cutter 300 to be the first thing that contacts the tissue and/or bone resulting in superior geometric positioning within the joint and more aggressive tissue and/or bone cutting.

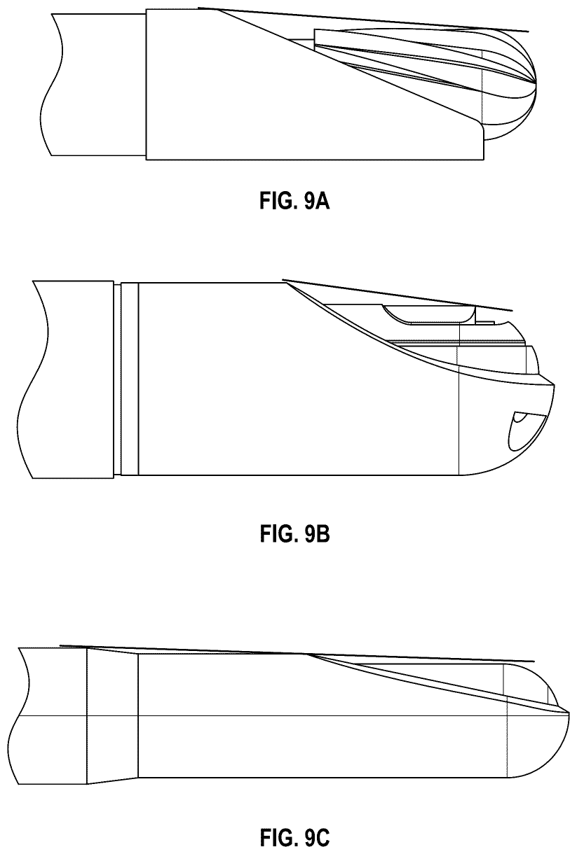

FIGS. 9A-9C show typical bone burr or bone shaver devices, which all include a negative angle of approach to tissue (i.e., the outer surface or edges of the cutters shown in FIGS. 9A-9C can form an acute declined angle with an outer surface of an outer shaft as shown with lines. Such arrangements of these devices require larger access and/or more manipulation to achieve desired resections. These devices also must be more aggressively manipulated to achieve desired bone cutting as compared with the devices of FIGS. 1-8B.

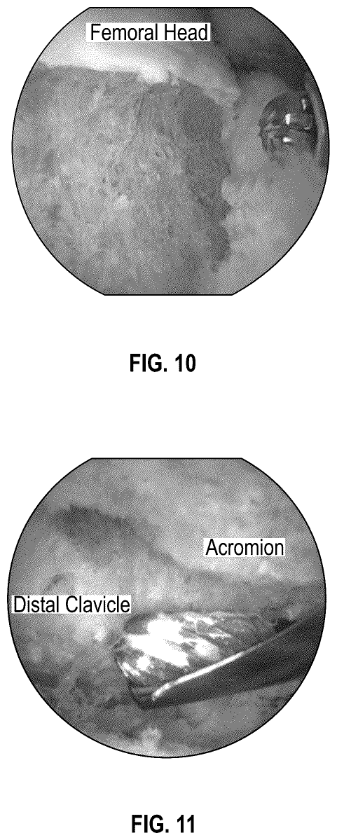

FIGS. 10 and 11 illustrate difficulties of manipulating a device with a negative angle of approach to tissue such as one of the cutters of FIGS. 9A-9C. In FIG. 10, an interior of the hip joint is shown with the tool positioned during a typical hip arthroscopy. The burr is working at a difficult angle of the shaft to the tissue at the distal tip (most difficult access) for the device. This arrangement, although typical in a hip arthroscopy, is different from a typical knee or shoulder where cutting is well angled (easy access) or parallel (medium difficult access) to the device shafts.

FIG. 11 illustrates a shoulder arthroscopy, which although not as difficult to access as a hip arthroscopy, still represents a difficult anatomical challenge for typical bone shavers and burrs as resection that is nearly parallel with the device shaft. In FIG. 11, the device position relative to the two bony surfaces that need to be resected (the acromion and the distal clavicle) are nearly parallel to the device shaft. This makes resection difficult because the cutting element is recessed relative to the outer shaft.

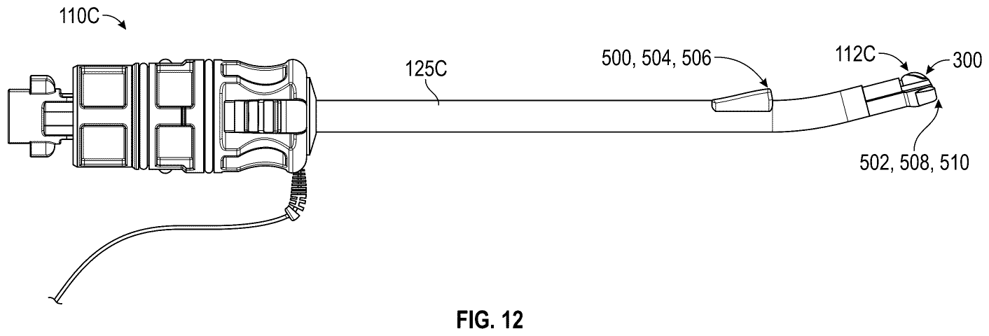

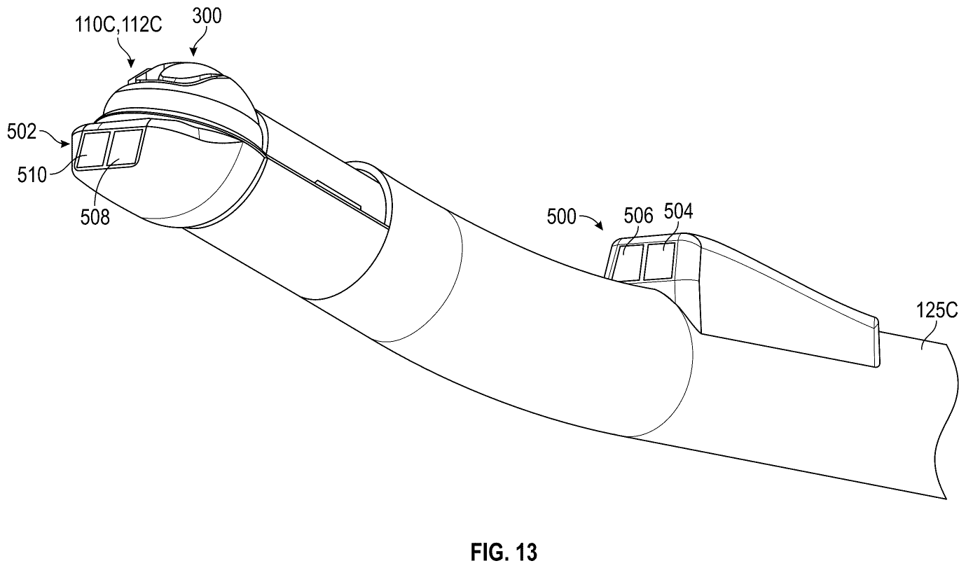

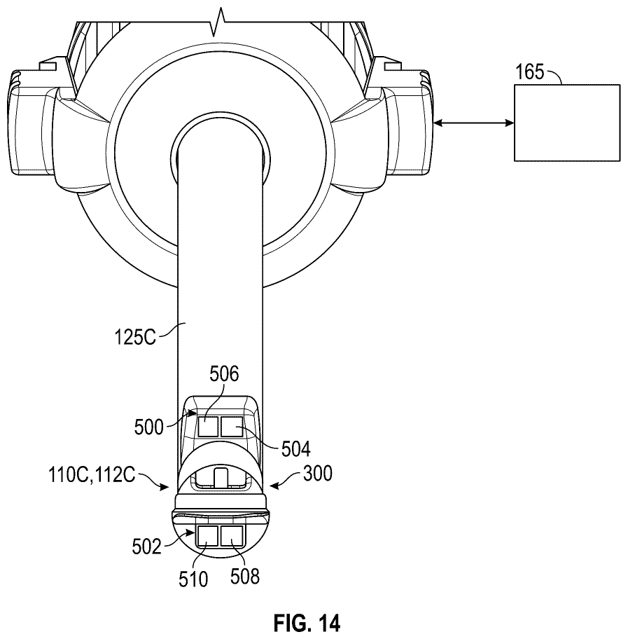

FIG. 12 shows a probe 110C with a shaft 125C and distal end 112C having a cutter 300 as previously discussed. FIGS. 13 and 14 are enlarged perspective views of the distal end 112C of the shaft 125C of the probe 110C with the cutter 300. The probe 110C differs from the probes discussed previously in that the probe 110C include a first visualization assembly 500 and a second visualization assembly 502. The first visualization assembly 500 can include one or more cameras 504 and one or more light sources 506. The second visualization assembly 502 can include one or more cameras 508 one or more light sources 510. FIG. 14 additionally schematically represents the controller 165 discussed previously as part of the system of FIG. 1.

The first visualization assembly 500 can be coupled to the proximal portion of the shaft 125C (here the outer shaft 302). The second visualization assembly 502 can be coupled to the distal tip portion 306. The first visualization assembly 500 and the second visualization assembly 502 can be utilized in tandem, toggled (switched) or operated in various different modes as discussed herein.

As shown in FIGS. 12-14, the light source 506 and the light source 510 can include one or more light emitting diodes (LEDs) or other illumination components. The LED can be an on-chip controlled devices with the chip being at or adjacent the first and/or second visualization assembly 500 and/or 502 or part of the probe 110C or handle. Thus, in some cases the chip can be at the distal end 112C or at another location on the probe 110C. This allows the LED to be controlled via actuator or other interface on the handle, for example. The one or more cameras 504 and/or 508 can be an on-chip controlled devices with the chip being at or adjacent the first and/or second visualization assembly 500 and/or 502 or part of the probe 110C or handle. This allows the one or more cameras 504 and/or 508 to be controlled via actuator or other interface on the handle, for example. For example, the one or more cameras 504 and/or 508 can utilize Complementary Metal-Oxide Semiconductor Active Pixel Sensors (CMOS-APS). The CMOS-APS are configured for sensing at an infrared wavelength range, a visual light wavelength range or another wavelength range as desired.

The light source 506 and the light source 510 can be identical or can differ from one another (e.g., the light source 506 can be configured for colored illumination while the light source 510 can be configured for white light illumination, the light source 506 can be brighter and/or differently arranged then the light source 510, etc.). Similarly, the one or more cameras 504 and the one or more cameras 508 can be identical or can differ from one another (e.g., the one or more cameras 504 can be configured for infrared sensing while the one or more cameras 508 can be configured for visual light sensing, etc.). The one or more cameras 508 can be angled relative to a longitudinal axis of the shaft 125C. Thus, the one or more cameras 508 can be at 0 degrees (aligned with) the longitudinal axis or angled at between 0.1 degrees and 90 degrees to the longitudinal axis. Similarly, the one or more cameras 504 can be angled relative to a longitudinal axis of the shaft 125C. Thus, the one or more cameras 504 can be at 0 degrees (aligned with) the longitudinal axis or angled at between 0.1 degrees and 90 degrees to the longitudinal axis. The light source 506 and/or the light source 510 can be angled relative to the longitudinal axis in a manner identical to or differing from the one or more cameras 504 and/or the one or more cameras 508.

The light source 506 and the light source 510 can be selectively controlled. Thus, light source 506 and the light source 510 can be activated at a same time, can be activated separately, a brightness of each of the light source 506 and the light source 510 can be controlled independently, etc. The light source 506 and/or the light source 510 can be configured to selectively increase or decrease in luminance, change illumination color, etc. under control such as actuated by the buttons on the handpiece as contemplated herein. The light source 506 and the light source 510 can be activated in sequence over a period of time. The light source 506 and the light source 510 can be activated to produce light at the same time but with the light source 506 having a brightness that differs from a brightness of the light source 510.

The one or more cameras 504 and the/or the light source 506 can be positioned so as to have a field of view that includes at least a part of the cutter 300. This arrangement allows the one or more cameras 504 and the/or the light source 506 to cast light and view the cutter 300 during cutting and/or coagulation. In contrast, the second visualization assembly 502 can be used to image capture and illuminate distal of the distal end 112C (distal of the distal tip) so as to image and illuminate the target tissue before cutting and/or coagulation. The second visualization assembly 502 has a location that can provide a traditional “endoscope” view of the joint. Thus, the first visualization assembly 500 can have the field of view that includes at least a portion of the cutter 300, according to some examples.

The one or more cameras 504 and the one or more cameras 508 can be selectively controlled. According to one example, the one or more cameras 504 and the one or more cameras 508 can be active/operational at the same time and can have the same focal point. Because the one or more cameras 504 and the one or more cameras 508 are at different distances with the same focal point a 3D composite image can be captured for processing. According to another example the field of view of the one or more cameras 504 can be to the left in FIG. 14 and the field of view of the one or more cameras 508 can to the right providing an overall 180 degree field of view. Thus, the first visualization assembly 500 can have a first field of view that can be offset to a first lateral side of a longitudinal axis of the outer shaft 302 and the second visualization assembly 502 can have a second field of view that can be offset to a second opposing lateral side of the outer shaft 302.

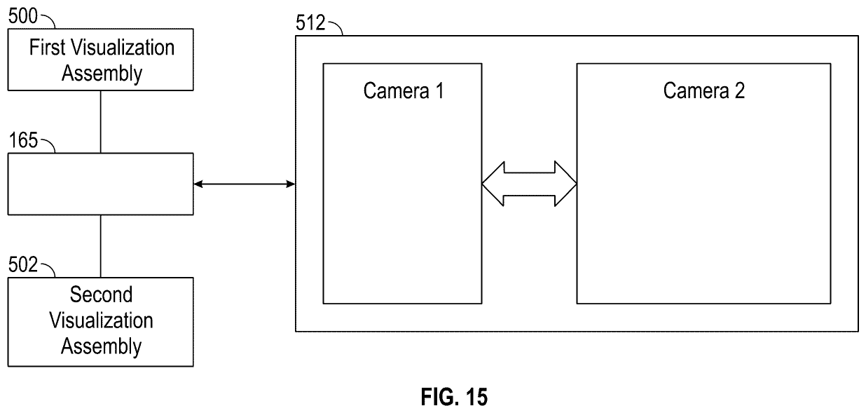

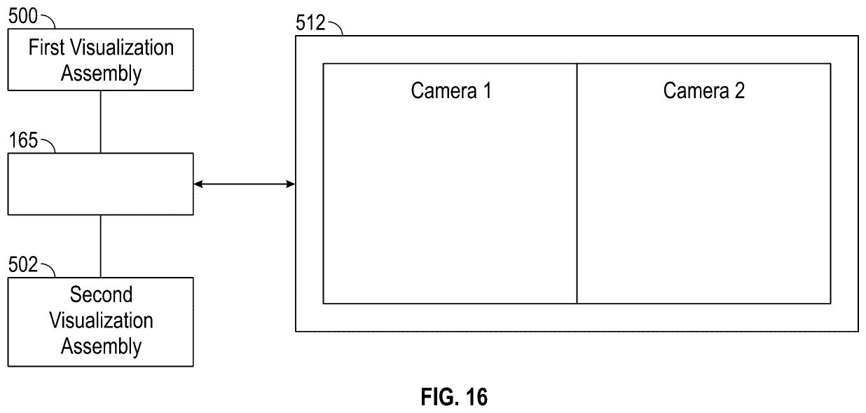

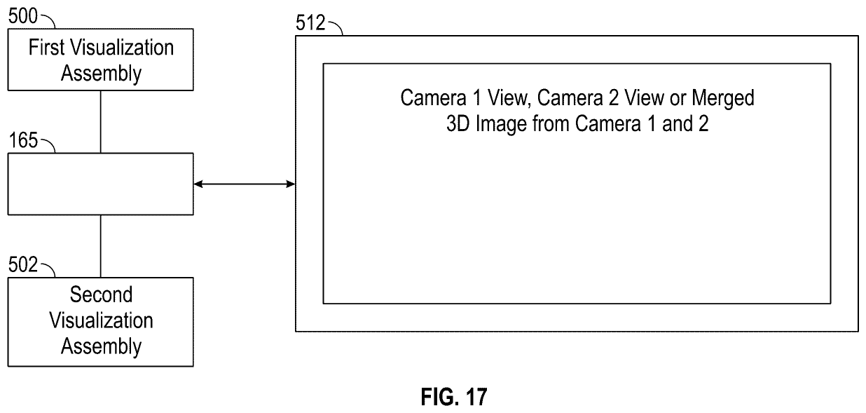

FIGS. 15-17 show the controller 165, the first visualization assembly 500, the second visualization assembly 502 and a display apparatus 512. The controller 165 can be electronically coupled to the display apparatus 512, the first visualization assembly 500 (also shown in FIGS. 12-14) and the second visualization assembly 502 (also shown FIGS. 12-14). The controller 165 can be configured control the first visualization assembly 500 and the second visualization assembly 502 and the display apparatus 512 to cause the display apparatus 512 to at least one of: change between the first field of view and the second field of view (as shown in FIG. 15), provide both the first field of view and the second field of view simultaneously (as shown in FIG. 16) or provide a three-dimensional composite view based upon the first field of view and the second field of view sharing substantially a same focal point (example of FIG. 17). Thus, the controller 165 can be configured to toggle between the two camera views of the first visualization assembly 500 and the second visualization assembly 502 and/or the controller 165 can be configured to provide both camera views from the first visualization assembly 500 and the second visualization assembly on the display apparatus 512 at the same time.

Alternatively, FIG. 15 or 17 illustrates that the change between the first field of view and the second field of view can be determined by the controller 165 based upon an operation mode of the medical device (the probe). In particular, the controller 165 can automatically switch between camera views from the first visualization system 500 and the second visualization system 502 based on device function. For example, during the diagnostic phase of the procedure the second visualization system 502 can be used and during activation phase (mechanical cutting, coagulation, ablation etc.) the camera view will automatically switch to the first visualization system 500 such that operation of the cutter can be monitored.

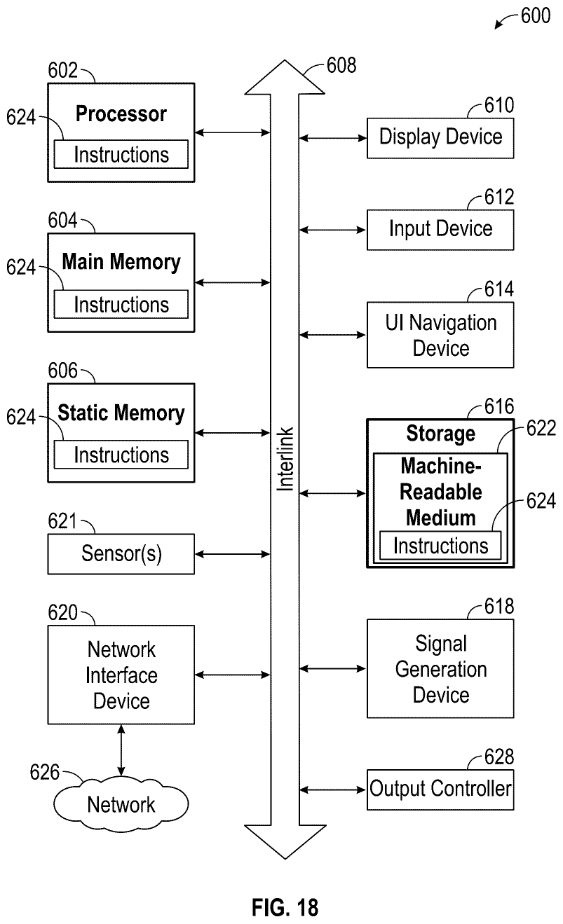

FIG. 18 illustrates a block diagram of an example machine 600 upon which any one or more of the techniques discussed herein may perform in accordance with some embodiments. This example machine can operate some or all of the apparatus and/or system function discussed herein. In other examples, the example machine 600 is merely one of many such machines utilized. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and plurality of sensors 621, such as any of those discussed previously (e.g., an IMU, a global positioning system (GPS) sensor, compass, accelerometer, or other sensor). The machine 600 may include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “substantially”, “generally” or “about” mean within 15% of the value provided. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Method and system examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

All references, including publications, patent applications, and patents, cited herein are hereby incorporated by reference to the same extent as if each reference were individually and specifically indicated to be incorporated by reference and were set forth in its entirety herein.

What is claimed is:

1. A medical device for removing tissue of a patient, comprising:
an outer shaft having a lumen, a proximal portion and an distal tip portion with an enlarged dimension relative to a diameter of the proximal portion; and
a cutter configured to remove the tissue, wherein the cutter is at least partially received by the distal tip portion and has an enlarged dimension relative to the diameter of the proximal portion of the outer shaft.

2. The medical device of claim 1, wherein the cutter is configured to provide the medical device with a positive approach angle to the tissue with an outer surface or one or more edges of the cutter forming an acute inclined angle with an outer surface of the outer shaft, wherein the cutter is rotatable relative to the outer shaft and includes a first side with an electrode having a radius of curvature, wherein the electrode is configured to provide radiofrequency energy to the tissue, and a second side with one or more features for mechanically cutting the tissue, wherein the one or more features include a plurality of resection teeth along a first edge of the second side and a blade having a radius of curvature along a second opposing edge of the second side.

3. The medical device of claim 2, wherein the cutter forms an opening to the lumen, wherein the opening has a maximum dimension that is smaller than a cross-sectional area of the lumen.

4. The medical device of claim 2, wherein the outer shaft at the distal tip portion has a hemispherical dish shape.

5. The medical device of claim 2, wherein the cutter has a radius of curvature along substantially an entirety of an outer surface thereof.

6. The medical device of claim 2, wherein the cutter has a truncated spheroid shape along an outer surface thereof.

7. The medical device of claim 1, wherein the cutter is nested in and captured by the distal tip portion such that the cutter is substantially prevented from proximal-distal movement relative to the distal tip portion of the outer shaft.

8. The medical device of claim 1, further comprising:
a first visualization assembly coupled to the proximal portion of the outer shaft, wherein the first visualization assembly includes one or more cameras; and
a second visualization assembly coupled to the distal tip portion, wherein the second visualization assembly includes one or more cameras.

9. The medical device of claim 8, wherein at least one of the first visualization assembly and the second visualization assembly includes a light source.

10. The medical device of claim 8, wherein the first visualization assembly has a first field of view that includes at least a portion of the cutter and the second visualization assembly has a second field of view that extends distal of the distal tip portion.

11. The medical device of claim 8, wherein the first visualization assembly has a first field of view is offset to a first lateral side of a longitudinal axis of the outer shaft and the second visualization assembly has a second field of view that is offset to a second opposing lateral side of the outer shaft.

12. The medical device of claim 10, further comprising:
a display apparatus; and
a controller electronically coupled to the display apparatus, the first visualization assembly and the second visualization assembly, wherein the controller is configured control the first visualization assembly and the second visualization assembly and the display apparatus to cause the display apparatus to at least one of: change between the first field of view and the second field of view, provide both the first field of view and the second field of view simultaneously or provide a three-dimensional composite view based upon the first field of view and the second field of view sharing substantially a same focal point.

13. The medical device of claim 12, wherein the change between the first field of view and the second field of view is determined by the controller based upon an operation mode of the medical device.

14. The medical device of claim 13, wherein the first visualization assembly includes a first one or more light sources and the second visualization assembly includes a second one or more light sources, wherein the controller is configured to control the first one or more light sources and the second one or more lights sources to be one or more of:
activated to produce light at a same time;
activated in sequence over a period of time;
activated to produce light at the same time but with the first one or more light sources having a brightness that differs from a brightness of the second one or more light sources.

15. A medical device for removing tissue of a patient, comprising:
an outer shaft having a lumen, a proximal portion and a distal tip portion with a hemispherical dish shape that has a dimension that is enlarged relative to a diameter of the proximal portion; and
a cutter configured to remove the tissue, wherein the cutter is at least partially nested in the distal tip portion and captured by the distal tip portion such that the cutter is substantially prevented from proximal-distal movement relative to the distal tip portion of the outer shaft.

16. The medical device of claim 15, wherein the cutter has an enlarged dimension relative to a diameter of the proximal portion of the outer shaft, wherein the cutter is configured to provide the medical device with a positive approach angle to the tissue with an outer surface or one or more edges of the cutter forming an acute inclined angle with an outer surface of the outer shaft, wherein the cutter is rotatable relative to the outer shaft and includes a first side with an electrode having a radius of curvature, wherein the electrode is configured to provide radiofrequency energy to the tissue, and a second side with one or more features for mechanically cutting the tissue, wherein the one or more features include a plurality of resection teeth along a first edge of the second side and a blade having a radius of curvature along a second opposing edge of the second side, wherein the cutter forms an opening to the lumen, wherein the opening has a maximum dimension that is smaller than a cross-sectional area of the lumen.

17. The medical device of claim 16, further comprising:
a first visualization assembly coupled to the proximal portion of the outer shaft, wherein the first visualization assembly includes one or more cameras; and
a second visualization assembly coupled to the distal tip portion, wherein the second visualization assembly includes one or more cameras.

18. The medical device of claim 17, further comprising:
a display apparatus; and
a controller electronically coupled to the display apparatus, the first visualization assembly and the second visualization assembly, wherein the controller is configured control the first visualization assembly and the second visualization assembly and the display apparatus to cause the display apparatus to at least one of: change between a first field of view a the second field of view, provide both the first field of view and the second field of view simultaneously or provide a three-dimensional composite view based upon the first field of view and the second field of view sharing a same focal point, wherein the change between the first field of view and the second field of view is determined by the controller based upon an operation mode of the medical device.

19. A medical device for removing tissue of a patient, comprising:
an outer shaft having a lumen, a proximal portion and a distal tip portion with an enlarged dimension relative to a diameter of the proximal portion; and
a cutter configured to remove the tissue, wherein the cutter is at least partially received by the distal tip portion and is configured to provide the medical device with a positive approach angle to the tissue with an outer surface or one or more edges of the cutter forming an acute inclined angle with an outer surface of the outer shaft, wherein the cutter has an enlarged dimension relative to a diameter of the proximal portion of the outer shaft.

20. The medical device of claim 19, wherein the cutter is rotatable relative to the outer shaft and includes a first side with an electrode having a radius of curvature, wherein the electrode is configured to provide radiofrequency energy to the tissue, and a second side with one or more features for mechanically cutting the tissue, wherein the one or more features include a plurality of resection teeth along a first edge of the second side and a blade having a radius of curvature along a second opposing edge of the second side, wherein the cutter forms an opening to the lumen, wherein the opening has a maximum dimension that is smaller than a cross-sectional area of the lumen, wherein the cutter has a radius of curvature along substantially an entirety of an outer surface thereof, wherein the cutter has a truncated spheroid shape along the outer surface thereof.
